(19) United States
(12) Patent Application Publication
(10) Pub. No.: US 2015/0366908 A1
(43) Pub. Date: Dec. 24, 2015

(54) SILICA-COATED CALCIUM SALT COMPOSITIONS

(71) Applicant: NOVABONE PRODUCTS, LLC, Alachua, FL (US)

(72) Inventors: Gregory J. Pomrink, Newberry, FL (US); Cecilia A. Cao, Gainesville, FL (US)

(73) Assignee: NOVABONE PRODUCTS, LLC, Alachua, FL (US)

(21) Appl. No.: 14/842,499

(22) Filed: Sep. 1, 2015

Related U.S. Application Data
(63) Continuation-in-part of application No. 13/912,490, filed on Jun. 7, 2013.
(60) Provisional application No. 61/656,741, filed on Jun. 7, 2012.

(51) Int. Cl. A61K 33/42; A61K 9/16; A61K 33/08

(52) U.S. Cl. CPC A61K 33/42; A61K 33/08; A61K 9/1617; A61K 9/1611; A61K 9/167

(57) ABSTRACT

A composition including calcium salt and silica, wherein the silica is in the form of a silicate that is adsorbed onto the surface of the calcium salt, wherein the silica is not incorporated into the structure of the calcium salt, and wherein the composition is bioactive.

### CROSS-REFERENCE TO RELATED APPLICATIONS

This application is a continuation-in-part application of U.S. patent application Ser. No. 13/912,490, filed Jun. 7, 2013, which claims the benefit of U.S. Provisional Patent Application No. 61/656,741, filed Jun. 7, 2012, the entire contents of which are hereby incorporated herein by reference.

### BACKGROUND OF THE INVENTION

There are many materials used today for the repair and regeneration of bone defects. Bone is a composite of collagen, cells, calcium hydroxyapatite crystals, and small quantities of other proteins of organic molecules that has unique properties of high strength, rigidity, and ability to adapt to varying loads. When bone injuries occur, it is necessary to fill voids or gaps in the bone as well as to encourage the repair and regeneration of bone tissue. Calcium salts are useful to fill voids and to encourage repair and regeneration.

There are significant drawbacks to the use of uncoated calcium salts to treat bone defects. Beta-tricalcium phosphate and calcium sulfate, for instance, degrade so quickly that the material is not suitable for treating load-bearing bones and in some cases may lead to insufficient bone formation. Uncoated calcium borate, for instance, releases borate ions into the matrix surrounding the material at too rapid of a rate to be of therapeutic benefit. Further, uncoated calcium salts are generally osteoconductive and not as effective as osteoinductive materials for the promotion of bone repair.

These drawbacks may be reduced and/or eliminated by coating calcium salts with a silica such that the rate of degradation is significantly reduced and that the calcium salts are no longer osteoconductive and osteoinductive.

### SUMMARY OF THE INVENTION

One embodiment provides for a composition comprising calcium salt and silica that is bioactive. The silica is in the form of an inorganic or organic silicate, i.e., with anionic or cationic moieties for complex formation with drug components that is adsorbed onto the surface of the calcium salt. The silica is not incorporated into the structure of the calcium salt.

Another embodiment provides for a method to stimulate osteoblast differentiation. An osteoblast is contacted with a composition comprising calcium salt and silica that is bioactive, as described above.

Another embodiment provides for a method to stimulate osteoblast proliferation. An osteoblast is contacted with a composition comprising calcium salt and silica that is bioactive, as described above.

Another embodiment provides for a method to regenerate bone. The region of bone at or near a site of a bone defect is contacted with the above-described composition comprising calcium salt and silica.

Another embodiment provides for a method to achieve critical concentrations of calcium ions and silicate ions in a bone defect. The region of bone at or near a site of the bone defect is contacted with the above-described composition comprising calcium salt and silica.

A further embodiment relates to a composition comprising calcium salt, silica and a metallic material having an atomic mass greater than 45 and less than 205, wherein the silica is in the form of a silicate that is adsorbed onto a surface of the calcium salt, wherein the silica is not incorporated into the structure of the calcium salt. The silica may be an organosilane, a sol-gel composition, a solution of silicated salt, a combination thereof or other silica-containing composition. The metallic material may be incorporated into the silica or may be a separate coating. In certain embodiments, the surface of the silica-coated calcium salt may be coated. In other embodiments, the metallic material coating may be applied prior to the application of the silica. The coating may be partial or complete. The metallic material may be selected, for example, from gold, silver, platinum, copper, palladium, iridium, strontium, cerium, or isotopes, or alloys thereof. The metallic material may be physically (van der Waal forces, or hydrogen-bonding) or chemically (covalent bonds) bound to the silica-coated calcium salt. The weight ratio of metallic material may be about 0.001%-20% relative to the weight of the composition. Alternatively, the weight ratio of the metallic material may be less than about 20%. The composition is osteoinductive and is capable of conducting an electrical current. The composition promotes more rapid wound healing as compared to a composition having uncoated silica-coated calcium salt. In case of metallic coating, the metallic material coating mount ranges from about 1 nm to about 1000 nm in thickness. In certain embodiments, the metallic material coating may be a dusting of the metallic material. The coating may be uniform or non-uniform. The composition may further include magnesium chloride or silica at least partially applied over the metallic material coating. The composition may further include a sol-gel glass coating at least partially applied over the metallic material coating. The composition may, further include an adhesive to aid in adhesion of the metallic material to the silica-coated calcium salt. The adhesive may be zirconium, titanium, chromium, or oxides thereof, other similar materials, and/or combinations thereof.

Yet further embodiment relates to a composition comprising calcium salt, silica and a metallic material having an atomic mass greater than about 45 and less than about 205, wherein the silica is in an organic or inorganic form selected from the group consisting of an organosilane, a sol-gel composition, a solution of silicated salt, a combination thereof or other silica-containing composition and is adsorbed only onto a surface of the calcium salt, wherein the silica is not incorporated into the structure of the calcium salt, and wherein the composition is bioactive. The organosilane may be γ-methacryloxypropyltrimethoxysilane, (3-glycidoxypropyl)-dimethyl-ethoxysilane, partially hydrolyzed tetraethyl orthosilicate, Silbond, 4-aminobutyltriethoxysilane, (3-aminopropyl)-triethoxysilane, (3-aminopropyl)-diethoxy-methylsilane, (3-aminopropyl)-dimethyl-ethoxysilane, (3-aminopropyl)-trimethoxysilane, (3-mercaptopropyl)-trimethoxysilane, or a combination thereof. The composition also comprises a metallic material. The metallic material may be gold, silver, platinum, copper, palladium, iridium, strontium, cerium, an isotope, an alloy or a combination thereof. The weight ratio of the metallic material is about 0.001%-20% relative to the weight of the composition; or is about 0.001%-10% relative to the weight of the composition. The composition conducts an electrical current. The metallic material may be dispersed into the silica glass. Alternatively, the metallic material forms a coating on the surface of the calcium salt. Alternatively, the metallic material forms a coating over the silica that is adsorbed onto the surface of the calcium salt.

### DETAILED DESCRIPTION OF THE INVENTION

One embodiment provides for a composition comprising calcium salt and silica that is bioactive. The silica is in the form of an organic and/or inorganic silicate that is adsorbed onto the surface of the calcium salt. The calcium salt is not substituted with silica.

In some embodiments, the calcium salt is calcium carbonate. The calcium carbonate may be at least 95% pure, at least 96% pure, at least 97% pure, at least 98% pure, or at least 99% pure. Such purified forms of calcium carbonate may be produced from a variety of sources of calcium carbonate, such as from a quarry, chalk, limestone, marble, or travertine. Calcium carbonate having the structural geometry of that found in coral may also be used. Methods of preparing purified calcium carbonate are known in the art, as there are many pharmaceutical forms of calcium carbonate already in use in the fields of toothpaste preparation, antacids, and calcium supplements. Various forms of pharmaceutical-grade calcium carbonate are also available and may be used. It is known in the art that precipitated and/or purified calcium carbonate has many different shapes and sizes of particles. The calcium carbonate salt may be in the form of a particle or pellet. The particle may have a mean size of 10 microns (μm) to 10 mm, 100 microns to 1 mm, 500 microns to 1.5 mm, 1 mm to 2 mm, or 1 mm to 3 mm. Among the various shapes, spindle-shaped calcium carbonate allows for efficient adhesion of a silica layer.

In some other embodiments of this aspect, the calcium salt is calcium borate. All bioactive calcium borates may be used. The calcium borate may be at least 95% pure, at least 96% pure, at least 97% pure, at least 98% pure, or at least 99% pure. One way of preparing calcium borate is to react calcium metal with boric acid. Calcium borate may also be obtained from various minerals, such as nobleite and priceite. The calcium borate salt may be in the form of a particle. The particle may have a mean size of 10 microns (μm) to 10 mm. Methods of preparing purified calcium borate are known in the art, as calcium borate finds application in the production of boron glasses.

In some embodiments, the calcium salt is calcium sulfate. All bioactive calcium sulfates may be used. Calcium sulfate may be at least 85% pure, at least 95% pure, at least 96% pure, at least 97% pure, at least 98% pure, or at least 99% pure. Calcium sulfate may be in various forms, such as the anhydrous form, the natural state, alpha-hemihydrate crystalline state, and the beta-hemihydrate crystalline state. Calcium sulfate may be prepared from gypsum and anhydrite. Methods of preparing purified calcium sulfate are known in the art, as calcium sulfate is used as a filler or excipient in the food and pharmaceutical industry. Various forms of pharmaceutical-grade calcium sulfate are also available and may be used. The calcium sulfate salt may be in the form of a particle. The particle may have a mean size of 10 microns (μm) to 10 mm.

In some embodiments, the calcium salt is calcium phosphate. All forms of bioactive calcium phosphate may be used including, for example, hydroxyapatite and beta calcium triphosphate. Calcium phosphate may be at least 85% pure, at least 95% pure, at least 96% pure, at least 97% pure, at least 98% pure, or at least 99% pure. Calcium phosphate may be prepared from bone meal or cow's milk, among other sources or synthesized from calcium salts and phosphoric acid. Methods of preparing purified calcium phosphate are known in the art. Various forms of pharmaceutical-grade calcium phosphate are available and may be used. In addition, various forms of calcium phosphate used in dental applications may be used. The calcium phosphate salt may be in the form of a particle. The particle may have a mean size of 10 microns (μm) to 10 mm.

In some embodiments, the calcium salt is beta calcium triphosphate (beta-TCP). Beta-TCP may be at least at least 85% pure, 95% pure, at least 96% pure, at least 97% pure, at least 98% pure, or at least 99% pure. It is known in the art that beta-TCP is readily available in the form of a synthetic bone grafting material. Beta-TCP may be in the form of a particle. The particle may have a mean size of 10 microns (μm) to 10 mm.

In some embodiments mixtures of calcium carbonate, calcium borate, calcium phosphate and/or other calcium salts may be used. The calcium salts may be at least at least 85% pure, 95% pure, at least 96% pure, at least 97% pure, at least 98% pure, or at least 99% pure.

The composition of any of the above embodiments may be osteoinductive. Osteoinduction allows for undifferentiated mesenchymal precursor cells to differentiate into bone forming cells. Osteoinductive compositions promote such differentiation. Bone morphogenetic proteins and osteogenic proteins such as collagen and osteonectin that are present in the extracellular matrix contribute to bone repair and regeneration. LeGeros, R. Z. describes the osteoinductive properties of calcium phosphate-based materials in Chem Rev. 2008, Vol. 108, pp. 4742-4753 and any of the materials described in that article may be used. Silicated calcium borate is osteoinductive for at least the reasons that silica reduces the pH of the environment around the calcium borate particles. Calcium carbonate having the structural geometry of that found in coral may also be used as an osteoinductive composition as it is known in the art that the structural geometry of coral and bone are similar.

In various embodiments, silica is applied to the calcium salts by spraying tetraethyl silicate (TEOS) or other silicates in ethanol with catalytic amounts of a volatile organic acid (i.e. acetic acid) and water over calcium salt granules (such as beta-TCP) while slowly mixing to continuously provide fresh uncoated (granule) surfaces for application (of the TEOS). The TEOS in ethanol solution may comprise TEOS:ethyl alcohol:acetic acid:water in a weight ratio of 10:8:1:1. Additional materials may be added to the oranganosilane solution including monovalent, divalent, and trivalent metal ions along with anionic species (e.g., carbonates, borates, titanates, zirconates). With regard to spraying, various proportions of calcium phosphate and TEOS in ethanol may be combined, such as by spraying a specific quantity of TEOS onto a specific quantity of calcium phosphate. Coating does not involve use of a silicate salt or bicalcium phosphate. The coated calcium salt may then dried under vacuum at room temperature or in a conventional oven at 50° C. Drying in a conventional oven may be undertaken for about one week to allow for evaporation of ethanol and acetic acid. Analysis of the dried material may be undertaken, such as by FTIR and/or ICP-MS, to determine the amount of silica. The finished silica coating on the calcium salt is durable and effective to reduce the rate of calcium ion transfer from the salt particle.

Alternatively, the silica may be applied by dipping calcium salt particles into tetraethyl silicate (TEOS). A change in mass of the TEOS solution may provide an indication as to the quantity of silica applied to the calcium salt particles. At the same time, analysis of the dried material may be undertaken, such as by FTIR and/or ICP-MS, to determine the amount of silica.

In various other embodiments, silica is applied to the calcium salts by spraying an anhydrous mixture of TEOS with a catalytic amount of a volatile organic acid followed by incubation under humid conditions (such as 60-80% relative humidity) for up to 24 hours followed by drying under vacuum at room temperature or in a conventional oven at 50° C.

Other organosilanes may be used in addition or in place of TEOS such as γ-methacryloxypropyltrimethoxysilane (hereinafter “A-174”), (3-glycidoxypropyl)-dimethyl-ethoxysilane (hereinafter “GPMES”), partially hydrolyzed TEOS, Silbond, and 4-aminobutyltriethoxysilane. Other silanization agents such as (3-aminopropyl)-triethoxysilane, (3-aminopropyl)-diethoxy-methylsilane, (3-aminopropyl)-dimethyl-ethoxysilane, (3-aminopropyl)-trimethoxysilane, and (3-mercaptopropyl)-trimethoxysilane, can also be used in addition to or in place of TEOS. There are numerous other silianes known to those of ordinary skill in the art that could be used, such as those currently sold by Gelest of Morrisville, Pa.

In some embodiments, a sol-gel bioactive glass could be used to coat the calcium salt particles. The organosilanes listed above may be used as the silica source. For example, a reaction mixture including tetraethoxysilane (TEOS), triethylphosphate (TEP), and calcium nitrate can be used to make sol-gel bioactive glasses. Other appropriate ingredients will also be apparent to those of ordinary skill in the art. Methods of preparing sol-gel reaction mixtures are well known as seen for example in U.S. Pat. No. 5,874,101 entitled “Bioactive-gel Compositions and Methods”, herein incorporated by reference in its entirety. Calcium salt containing particles can be coated by, for example, immersing the particles in the sol-gel reaction solution and pouring off the excess sol-gel reaction solution or spraying the sol-gel reaction solution on the surfaces of the particles. The coated particles may then be aged and/or dried.

In some embodiments, the calcium salts may be in the form of a ceramic. The ceramic may be formed from a ceramic precursor composition comprising calcium-silicate mineral. The ceramic may be cured before coating with silica. Alternatively, the ceramic may be coated with silica before curing.

In some embodiments, the silicate may also be at least partially covalently bonded to the calcium salt.

In various other embodiments, if a homogenously coated application is not required, direct mixing of the TEOS solution with the beta-TCP can be undertaken. A sufficient quantity of silica can be present to reduce the resorption rate of calcium and other ions back into the particle. The reduction in resorption rate is proportional to the amount of silica adsorbed onto the surface. The silica concentration may be in the range of from about 0.0001 molar to about 0.5 molar. In some alternatives, the ratio of silica and the composition is from 0.01 wt % to 50 wt %. In other alternatives, the ratio of silica and the composition is from 1 wt % to 5 wt % and 5 wt % to 25 wt %. The silica is effective to reduce the resorption rate of calcium sulfate and/or beta calcium triphosphate. The silica layer may also be used to control the diffusion of ions, such as calcium and phosphate, from the particles to the surface. Further, the silica layer may release silicon from the surface to stimulate bone cell function.

In some embodiments, the silicate is substituted with a functional group. Functional groups include one or more of quinolinol and hydroxyquinoline. Any number of substituted silanes may be used, such as those sold by Gelest Inc.

Another embodiment provides for a method to stimulate osteoblast differentiation. An osteoblast is contacted with a composition comprising calcium salt and silica that is bioactive, as described above. The osteoblast then undergoes differentiation.

Another embodiment provides for a method to deliver drugs to bone. A composition comprising calcium salt, silica, and a drug is contacted with bone. The drug is delivered to the bone.

Another embodiment provides for a method to bind proteins found in bone, such as BMP.

Another embodiment provides for a method to stimulate osteoblast proliferation. An osteoblast is contacted with a composition comprising calcium salt and silica that is bioactive, as described above. The osteoblast then proliferates. For example, DNA array studies by Hench et al. demonstrate that calcium and silica active genes are responsible for osteoblast differentiation and proliferation.

Another embodiment provides for a method to regenerate bone. The region of bone at or near a site of a bone defect is contacted with the above-described composition comprising calcium salt and silica. The composition may be secured to the bone by means of a bag, or coated on screws, posts, staples, pins, buttons, and combinations thereof. The bone anchoring device can be attached to a drilled or hollowed out region of bone.

Another embodiment provides for a method to achieve critical concentrations of calcium ions and silicate ions in a bone defect. The composition may be in the form of a putty, cement, composite, or other bone fill material. When calcium and silicate ions are provided by means of a sufficient number of calcium salt particles coated with silica, the concentrations of calcium and silicate increase to a critical level such that osteoblast differentiation and proliferation can occur. Such differentiation and proliferation can arise from stimulation of genes in the osteoblast that are responsible for such effects. The region of bone at or near a site of the bone defect is contacted with the above-described composition comprising calcium salt and silica. The composition may be secured to the bone by means of a bag, or coated on screws, posts, staples, pins, buttons, and combinations thereof. The bone anchoring device can be attached to a drilled or hollowed out region of bone. Drug delivery or protein binding for controlled release, such as cationic (PEI), has been shown to reduce the kinetics of BMP 2. Also components binding with polymers show increases in strength, such as A-174 with methacrylates. Antimicrobial agents or antibiotic agents may also be present in the compositions.

Further embodiments relate to compositions comprising calcium salt, silica and a metallic material having an atomic mass greater than about 45 and less than about 205, wherein the silica is in the form of a silicate that is adsorbed onto a surface of the calcium salt, wherein the silica is not incorporated into the structure of the calcium salt. The silica may be an organosilane, a sol-gel composition, a solution of silicated salt, a combination thereof or other silica-containing composition. The metallic material may be integrated with the silica or form a surface coating over or under the silica. In certain embodiments, the surface of the calcium salt or the silica-coated calcium salt may be partially coated. The metallic material may be selected, for example, from gold, silver, platinum, copper, palladium, iridium, strontium, cerium, or isotopes, or alloys thereof. The metallic material may be physically (van der Waal forces, or hydrogen-bonding) or chemically (covalent bonds) bound to the silica-coated calcium salt. The weight ratio of metallic material may be about 0.001%-20% relative to the weight of the composition. Alternatively, the weight ratio of the metallic material may be less than about 20%. The composition is osteoinductive and is capable of conducting an electrical current. The composition promotes more rapid wound healing as compared to a composition without the metallic material. The metallic material coating mount ranges from about 1 nm to about 1000 nm in thickness. In certain embodiments, the metallic material coating may be a dusting of the metallic material. The coating may be uniform or non-uniform. The composition may further include magnesium chloride or silica at least partially applied over the metallic material coating. The composition may further include a sol-gel glass coating at least partially applied over the metallic material coating. The composition may, further include an adhesive to aid in adhesion of the metallic material to the silica-coated calcium salt. The adhesive may be zirconium, titanium, chromium, or oxides thereof, other similar materials, and/or combinations thereof.

Metallic materials, such as gold, silver, platinum, copper, palladium, iridium, strontium, cerium, or isotopes, or alloys, or salts thereof, when incorporated (e.g., by coating, or integrating into the structure) into the composition comprising the silica-coated calcium salt are able to conduct an electrical current and prevent or reduce body's inflammatory response at or near the injury site upon the delivery of the composition comprising calcium salt, silica and a metallic material, enhancing the activity of the calcium salt and the bone healing process. When bone is injured, it generates an electrical field. This low-level electrical field is part of the body's natural process that stimulates bone healing. When this healing process fails to occur naturally, a conductive implant material can facilitate regeneration of the bone. Conductive implants provide a safe, treatment that helps promote healing in fractured bones and spinal fusions which may have not healed or have difficulty healing. The devices stimulate the bone's natural healing process by sending low-level pulses of electromagnetic energy to the injury or fusion site.

As such, coating bone grafting materials with a metallic material or otherwise incorporating the metallic materials into the bone grafting materials or compositions provides a solution to the problem of unwanted inflammatory response that may arise from an injury as well as the presence of calcium salt. Also, by having a metallic material, such as gold coated on the surface of the calcium salt (rather than incorporated into the structure of the material), the surfaces becomes conductive and the gold becomes available to function in reducing inflammation immediately upon the delivery of the metallic gold-coated silica-coated calcium salt.

Metallic materials, such as gold are known to be highly conductive and possess anti-inflammatory properties. Importantly, electrical conductance and reduction of inflammation at the site of a wound may increase the rate at which the wound heals. Metallic materials may also promote wound healing by initiating or promoting angiogenesis. Increased blood flow may increase the rate of wound healing. Other benefits of gold may also be present.

The term “metallic material” refers to pure metals, such as gold, silver, platinum, copper, palladium, iridium, strontium, cerium or isotopes (including radioisotopes), or alloys, or salts (the ionic chemical compounds of metals) thereof or other metallic materials having an atomic mass greater than about 45 and less than about 205. The term “atomic mass” is the mass of an atomic particle, sub-atomic particle, or molecule. It is commonly expressed in unified atomic mass units (u) where by international agreement, 1 unified atomic mass unit is defined as 1/12 of the mass of a single carbon-12 atom (at rest).

The term “metal alloy” refers to a material that's made up of at least two different chemical elements, one of which is a metal. The most important metallic component of an alloy (often representing 90 percent or more of the material) is called “the main metal,” “the parent metal,” or “the base metal.” The other components of an alloy (which are called “alloying agents”) can be either metals or nonmetals and they're present in much smaller quantities (sometimes less than 1 percent of the total). Although an alloy can sometimes be a compound (the elements it's made from are chemically bonded together), it's usually a solid solution (atoms of the elements are simply intermixed, like salt mixed with water). Examples of alloys include, e.g., bronze (copper (78-95%), tin (5-22%), plus manganese, phosphorus, aluminum, or silicon); amalgam (mercury (45-55%), plus silver, tin, copper, and zinc); steel (stainless; iron (50%+), chromium (10-30%), plus smaller amounts of carbon, nickel, manganese, molybdenum, and other metals), sterling silver (silver (92.5%), copper (7.5%)).

The term “metal isotopes” refers to variants of a particular chemical element which differ in neutron number, although all isotopes of a given element have the same number of protons in each atom. One example of a stable isotope of gold is gold-197(¹⁹⁷Au). Examples of isotopes of copper include copper-63 (⁶³Cu) and copper-65 (⁶⁵Cu); examples of isotopes of iridium include iridium-192 (¹⁸²Ir) and iridium-193 (¹⁹²Ir) examples of isotopes of palladium include palladium-102 (¹⁰²Pd), 104 (¹⁰⁴Pd), 105 (¹⁰⁵Pd), 106 (¹⁰⁶Pd), 108 (¹⁰⁸Pd) and 110 (¹¹⁰Pd) examples of isotopes of platinum include, e.g., five stable isotopes (¹⁹²Pt, ¹⁹⁴Pt, ¹⁹⁵Pt, ¹⁹⁶Pt, ¹⁹⁸Pt) and one very-long lived (half-life 6.50×10¹¹ years) radioisotope (¹⁹⁰Pt).; examples of isotopes of silver include two stable isotopes ¹⁰⁷Ag and ¹⁰⁹Ag with ¹⁰⁷Ag; examples of isotopes of strontium include four stable, naturally occurring isotopes: ⁸⁴Sr (0.56%), ⁸⁶Sr (9.86%), ⁸⁷Sr (7.0%) and ⁸⁸Sr (82.58%).

The term “metal salts” refers to the ionic chemical compounds of metals. For example gold salts include, e.g., sodium aurothiomalate and auranofin.

The terms “integrated” or “incorporated” refer to the metallic materials that may be included as part of the bone grafting composition either by coating the surface of the bone grafting composition or by including or integrating the metallic materials in the structure of the bone grafting composition.

In certain embodiments, at least a portion of the silica-coated calcium salt composition may be coated with a thin layer or film of metallic material such as gold; alternatively, substantially entire surface may be coated with a thin layer or film of metallic material. For example, when the silica-coated calcium salt is in a form of a particle, substantially entire surface of the particle is coated with a thin layer of gold. In another example, when the silica-coated calcium salt composition is in a form of a block of material or a composite, substantially entire outer surface of the block of material is coated with a thin layer of gold.

In certain other embodiments, the metallic materials may be integrated into the structure of the silica-coated calcium salt composition. For example, a metal salt or a metal particle can be dissolved into silica and used as a coating for bone grafting materials. In another example, metal particles can be dispersed (i.e., scattered, disseminated, distributed, spread) into the silica before the silica is absorbed onto the calcium salt.

In certain embodiments, the silica-coated calcium salt is coated with a thin layer of a film of metallic material such as gold without using an adhesion layer, such as chromium or titanium based adhesion layer.

In the compositions described herein, the metallic material may be present in approximate amounts of 0.001-20 wt. % ratio with reference to the total weight of the composition. Alternatively, the metallic material may be present in approximate amounts of 0.001-10 wt. % ratio with reference to the total weight of the composition. The metallic material may also be present in a weight ratio of less than 10 wt. %; less than about 5 wt. %; less than about 2.5 wt. %; less than about 1 wt. %; or less than about 0.5 wt. %. In some embodiments, the weight ratio may be about 0.1%, about 0.2%, about 0.3%, about 0.4%, about 0.5%, about 0.6%, about 0.7%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.5%, about 4%, about 4.5%, or about 5%.

In some embodiments, pure metals, metal alloys, metal isotopes or radioisotopes, or salts formed therefrom may be bound to the silica-coated calcium salt. The metallic material may be physically (van der Waal forces, or hydrogen-bonding) or chemically (covalent bonds) bound to the silica-coated calcium salt. Such bonding may occur by any means known to one skilled in the art, including but not limited to, the formation of covalent bonds, van der Waal forces, or hydrogen-bonding. Gold is utilized in the following specific examples to further illustrate the bone grafting compositions and should not be construed to limit the scope of the disclosure. The metals may include other precious metals without departing from or exceeding the spirit or scope of the disclosure. The surface of gold, gold alloys, and gold isotopes or radioisotopes may be functionalized with complexes or compounds that have carboxylic acid groups, hydroxyl groups, thiol groups, phosphate groups, or amide functional groups, to name a few, that can be used to form covalent bonds with silica-coated calcium salt through the use of a coupling agent. An exemplary coupling agent is aminopropyl silane. Such coupling agents are available from Gelest Inc., for example. Other coupling agents include amine, sulfur, phosphorus, epoxy, hydride and carboxylate agents. Specific examples of coupling agents include, but are not limited to, aminopropyl triethoxysilane, diaminopropyl diethoxysilane, glycidoxypropyl trimethoxysilane, aminopropyl trimethoxysilane, aminopropyl triethoxysilane, carboxyethylsilanetriol, triethoxysilylpropylmaleamic acid, N-(trimethoxysilyl propyl)ethylene diamine triacetic acid, 3-(trihydroxysilyl)-1-propane sulfonic acid, and 2-(4-chlorosulfonylphenyl)ethyltrimethoxysilane. Additional coupling agents include amine, sulfur, phosphorus, epoxy, hydride and carboxylate agents. When these coupling agents are used, the trialkoxy groups may directly react with the surface of the silica-coated calcium salt or hydrolyze to form hydroxyl groups that react with the surface of the silica-coated calcium salt through the formation of hydrogen bonds or covalent linkages, while the amino portion of the coupling agent interacts with the gold, gold alloys, salts or radioisotopes. The end result is the bonding of the gold, gold alloys, salts or radioisotopes to the silica-coated calcium salt.

As gold is a metal, in certain embodiments, it can form an alloy with other metals. For example, gold may form an alloy with silver, copper, rhodium, nickel, platinum, palladium, zinc, or aluminum, to name a few.

In various embodiments, the metallic materials, metallic material alloys, salts or radioisotopes need not remain bound to the silica-coated calcium salt after implantation of a metallic material-coated composition into the body. In the body, the gold may eventually be disassociated from the silica-coated calcium salt. The silica-coated calcium salt and the metallic material would both be present in the tissue near the implant site. Both substances can then promote healing of the wound at the implant site. The advantage of the metallic material such as gold being coated on the surface of the silica-coated calcium salt is that the gold becomes available immediately upon implantation to the body (rather than as the composition dissolves) to help with any anti-inflammatory response at the site of the implantation as well as around the site. Without being bound by any particular mechanism, the silica-coated calcium salt may promote bone repair and induce soft tissue repair by the release of calcium ions. The metallic material, e.g., gold, may promote immediately aid in reducing inflammation, and/or counteract any tendency of the silica-coated calcium salt in the wound site to promote coagulation, promote angiogenesis, and enhance soft tissue repair.

In any of the embodiments, the composition including metallic materials promotes more rapid wound healing than that achieved by non-conductive compositions including calcium salt and silica without metallic materials. The metallic material serves to conduct electrical current, reduce the inflammation and enhance the rate of wound healing. Further, conductivity of the implant material along with the ions released by the calcium salt combined with the activity of the gold may synergistically enhance the rate of wound healing. Synergy may arise from any one or more of the following metallic material activities: anti-inflammatory activity, reduction of blood clotting and/or coagulation, facilitation of the migration of cells into the scaffold, formation of blood vessels, and stimulation of genes to increase the rate of healing of hard and soft tissues.

Another embodiment relates to a method for treating a wound. A composition comprising calcium salt, silica and a metallic material, such as, e.g., gold is applied to the wound. The composition may be in the form of a particle, a glass sheet, a fiber, a mesh, block, wedge, strip, or other shape or a composition containing a composite of varying shape or size. The wound comprises one or more of a bone injury and a soft tissue injury. The composition is effective to accelerate repair of the bone injury and the soft tissue injury.

Another embodiment provides for a method of treating a bone defect. A composition comprising calcium salt, silica and a metallic material is applied to the site at or near the bone defect. The composition may be in the form of a particle, a glass sheet, a fiber, a block, a wedge, a strip, a mesh, or any combination of these forms. The coated composition is bioresorbable at a rate consistent with the rate of formation of new bone at or near the site.

Another embodiment provides for a method of preparing a composition comprising calcium salt, silica and a metallic material. A metallic material can be coated onto at least a portion of the surface of the calcium salt or the silica-coated calcium salt compositions by methods known in the art.

For example, one method includes coating by means of dipping or spraying the composition with a solution containing a metallic material. For example, the solution can be spray applied or poured onto/over the composition. Porous or non-porous blocks of silica-coated calcium salt composition can be dipped into a solution of metallic material. The silica-coated calcium salt can then be dried using a variety of techniques, including but not limited to freeze drying, vacuum drying, oven drying, and spray drying. The process can be repeated until the desired ratio of metallic material to silica-coated calcium salt is achieved.

Another method of coating with metallic materials includes sputter deposition, which is a physical vapor deposition (PVD) method of thin film deposition by sputtering. This involves ejecting material from a “target” that is a source onto a “substrate” such as silica-coated calcium salt. PVD includes a variety of vacuum deposition methods that can be used to deposit thin films of metallic material by the condensation of a vaporized form of metallic film material onto silica-coated calcium salt. The coating method involves purely physical processes such as high-temperature vacuum evaporation with subsequent condensation, or plasma sputter bombardment rather than involving a chemical reaction at the surface to be coated as in chemical vapor deposition.

Another method includes a sputter deposition process to cover the calcium salt or the silica-coated calcium salt with a thin layer of metallic material, such as, e.g., such as gold or a gold/palladium (Au/Pd) alloy.

In various embodiments, the metallic material need not remain bound to the bioactive glass ceramic material after implantation of a composition into the body. Preferably, in the body, the metallic material becomes immediately available for reducing inflammation at the implantation site. Without being bound by any particular mechanism, the metallic material may inhibit or reduce the inflammation, promote angiogenesis, enhance soft tissue repair, and/or counteract any tendency of the silica-coated calcium salt in the wound site to promote coagulation.

Throughout this specification various indications have been given as to preferred and alternative embodiments of the invention. However, the foregoing detailed description is to be regarded as illustrative rather than limiting and the invention is not limited to any one of the provided embodiments. It should be understood that it is the appended claims, including all equivalents that are intended to define the spirit and scope of this invention.

Other potential uses for the compositions described herein include their use in hemostasis, bone regeneration, soft and hard tissue repair, delivery of therapeutic agents, spine surgery, de-compressive craniotomy surgery, and treating iliac crest defects.

### EXAMPLES

### Example 1

### Silanation with TEOS-Spray Application Method

100 g of 1-2 mm calcium phosphate was added to a mixing bowl. A TEOS solution was prepared with 12.5 g TEOS, 10 g ethyl alcohol, 1.25 g acetic acid, and 1.25 g water and then poured into a spray bottle. The spray bottle was weighed and the weight was recorded.

A 1% silicate beta-TCP solution was prepared as follows. The TEOS solution was sprayed onto 100.00 mg calcium phosphate while the glass was continually mixed. After 2-3 sprays, the spray bottle was weighed and the change in weight was recorded such that the weight of solution per spray was roughly determined. Additional TEOS solution was sprayed onto the calcium phosphate until the weight of the spray bottle was reduced by 7.00 g. After the TEOS solution has been applied, the glass was mixed for an additional 5-10 minutes, with continuous scraping of the walls and the bottom of the bowl.

A lid was placed on the mixing bowl and the treated calcium phosphate was incubated in an oven for 120 hours at 50° C. Following incubation, the treated glass was poured onto a drying tray and placed back into the oven at 50° C. The glass was dried for 1 week at 50° C. to evaporate residual ethanol and acetic acid. The silicated TCP was removed from the oven. ICP-MS and FTIR scans for the material were obtained to determine the amount of silica present.

### Example 2

### Silanation with TEOS-Spray Application Method to Prepare Various Silicated TCP Formulations

TABLE 1

| | | | | |
|---|---|---|---|---|
| Material | % | MW | 25 g | 50 g |

TEOS Solution Formulation

| | | | | |
|---|---|---|---|---|
| TEOS | 50.00 | 208.33 | 12.5 | 25.00 |
| Ethyl Alcohol | 40.00 | 46.00 | 10 | 20.00 |
| Acetic Acid | 5.00 | 74.00 | 1.25 | 2.50 |
| Water | 5.00 | 18.00 | 1.25 | 2.50 |

Silicated TCP Formulations

| | | | | |
|---|---|---|---|---|
| wt % Coating | 0.1% | 1% | 3% | 5% |
| Calcium | 100.00 | 100.00 | 100.00 | 100.00 |
| Phosphate (g) | | | | |
| Solution (g) | 0.70 | 7.00 | 21.00 | 35.00 |

Various different silicated TCP formulations are prepared according to the method of Example 1. Table 1 shows the amounts of TEOS, ethyl alcohol, acetic acid, and water to use to prepare various weights of solution, e.g. 25 g and 50 g. The amounts may be scaled proportionally to prepare different weights of solution as well.

Table 1 also shows the amount of solution to be sprayed onto 100.00 g of calcium phosphate. For instance, to prepare 3% weight coating, 21.00 g of solution is sprayed onto 100.00 g of calcium phosphate. The amounts may be scaled proportionally to prepare different coating weights onto different amounts of calcium phosphate as well at, for example, 10, 15, 20 and 25 wt % coating.

### Example 3

### Silanation with TEOS-Soaking Method

100 g of 1-2 mm calcium phosphate was added to a mixing bowl. A TEOS solution was prepared with 12.5 g TEOS, 10 g ethyl alcohol, 1.25 g acetic acid, and 1.25 g water, with 7.00 g poured into a glass beaker. 100.00 g of TCP was then added to the beaker and soaked to prepare 1% silicated beta-TCP. The TCP in the TEOS solution was stirred until all of the particulate has been coated. A lid was then placed on the beaker and the calcium phosphate/TEOS mixture was then incubated in an oven for 120 hours at 50° C. Following incubation, the treated glass was poured onto a drying tray and placed back into the oven at 50° C. The glass was dried for one week at 50° C. to evaporate residual ethanol and acetic acid. The silicated TCP was removed from the oven. ICP-MS and FTIR scans were obtained for the material to determine the amount of silica present.

### Example 4

### Silanation with TEOS-Condensation Method

100 g of 1-2 mm calcium phosphate was weighed into a large crystallizing dish. Two small beakers were placed in the crystallizing dish, such that the lip of the beaker was below the lip of the crystallizing dish. One of the small beakers was filled with 20 mL of TEOS and the other small beaker was filled with 30 mL of RODI. Aluminum foil was placed over the crystallizing dish, which was incubated in the oven for 120 hours at 50° C. Following incubation, the treated glass was poured onto a drying tray and placed back into the oven at 50° C. for 1 week. The silicated TCP was removed from the oven. ICP-MS and FTIR scans were obtained for the material to confirm the amount of silica present.

### Example 5

### Silanation with GPMES

100 g of 1-2 mm calcium phosphate was added to a mixing bowl. A GPMES solution was prepared with 12.5 g GPMES, 10 g ethyl alcohol, 1.25 g acetic acid, and 1.25 g water and then poured into a spray bottle. The spray bottle was weighed and the weight was recorded.

A 1% silicate beta-TCP solution was prepared as follows. The GPMES solution was sprayed onto 100.00 mg calcium phosphate while the glass was continually mixed. After 2-3 sprays, the spray bottle was weighed and the change in weight was recorded such that the weight of solution per spray was roughly determined. Additional GPMES solution was sprayed onto the calcium phosphate until the weight of the spray bottle was reduced by 7.27 g. After the GPMES solution has been applied, the glass was mixed for an additional 5-10 minutes, with continuous scraping of the walls and the bottom of the bowl.

A lid was placed on the mixing bowl and the treated calcium phosphate was incubated in an oven for 120 hours at 50° C. Following incubation, the treated glass was poured onto a drying tray and placed back into the oven at 50° C. The glass was dried for 1 week at 50° C. to evaporate residual ethanol and acetic acid. The silicated TCP was removed from the oven. ICP-MS and FTIR scans for the material were obtained to determine the amount of silica present.

### Example 6

### Silanation with GPMES to Prepare Various Silicated TCP Formulations

TABLE 2

| | | | | |
|---|---|---|---|---|
| Material | % | MW | 25 g | 50 g |

(3-Glycidoxypropyl)dimethylethoxysilane (GPMES) Solution

| | | | | |
|---|---|---|---|---|
| GPMES | 50.00 | 218.37 | 12.5 | 25.00 |
| Ethyl Alcohol | 40.00 | 46.00 | 10 | 20.00 |
| Acetic Acid | 5.00 | 74.00 | 1.25 | 2.50 |
| Water | 5.00 | 18.00 | 1.25 | 2.50 |

Silicated TCP Formulations

| | | | | |
|---|---|---|---|---|
| wt % Coating | 0.1% | 1% | 3% | 5% |
| Calcium | 100.00 | 100.00 | 100.00 | 100.00 |
| Phosphate (g) | | | | |
| Solution (g) | 0.73 | 7.27 | 21.80 | 36.34 |

Various different silicated TCP formulations are prepared according to the method of Example 5. Table 2 shows the amounts of GPMES, ethyl alcohol, acetic acid, and water to use to prepare various weights of solution, e.g. 25 g and 50 g. The amounts may be scaled proportionally to prepare different weights of solution as well.

Table 2 also shows the amount of solution to be sprayed onto 100.00 g of calcium phosphate. For instance, to prepare 3% weight coating, 21.80 g of solution is sprayed onto 100.00 g of calcium phosphate. The amounts may be scaled proportionally to prepare different coating weights onto different amounts of calcium phosphate as well at, for example, 10, 15, 20 and 25 wt % coating.

### Example 7

### Silanation with A-174

100 g of 1-2 mm calcium phosphate was added to a mixing bowl. An A-174 solution was prepared with 12.5 g A-174, 10 g ethyl alcohol, 1.25 g acetic acid, and 1.25 g water and then poured into a spray bottle. The spray bottle was weighed and the weight was recorded.

A 1% silicate beta-TCP solution was prepared as follows. The A-174 solution was sprayed onto 100.00 mg calcium phosphate while the glass was continually mixed. After 2-3 sprays, the spray bottle was weighed and the change in weight was recorded such that the weight of solution per spray was roughly determined. Additional A-174 solution was sprayed onto the calcium phosphate until the weight of the spray bottle was reduced by 7.27 g. After the A-174 solution has been applied, the glass was mixed for an additional 5-10 minutes, with continuous scraping of the walls and the bottom of the bowl.

A lid was placed on the mixing bowl and the treated calcium phosphate was incubated in an oven for 120 hours at 50° C. Following incubation, the treated glass was poured onto a drying tray and placed back into the oven at 50° C. The glass was dried for 1 week at 50° C. to evaporate residual ethanol and acetic acid. The silicated TCP was removed from the oven. ICP-MS and FTIR scans for the material were obtained to determine the amount of silica present.

### Example 8

### Silanation with A-174 to Prepare Various Silicated TCP Formulations

TABLE 3

| | | | | |
|---|---|---|---|---|
| Material | % | MW | 25 g | 50 g |

Methacryloxypropyltriethoxysilane (A-174) Solution

| | | | | |
|---|---|---|---|---|
| A-174 | 50.00 | 290.43 | 12.5 | 25.00 |
| Ethyl Alcohol | 40.00 | 46.00 | 10 | 20.00 |
| Acetic Acid | 5.00 | 74.00 | 1.25 | 2.50 |
| Water | 5.00 | 18.00 | 1.25 | 2.50 |

Silicated TCP Formulations

| | | | | |
|---|---|---|---|---|
| wt % Coating | 0.1% | 1% | 3% | 5% |
| Calcium | 100.00 | 100.00 | 100.00 | 100.00 |
| Phosphate (g) | | | | |
| Solution (g) | 0.97 | 9.67 | 29.00 | 48.33 |

Various different silicated TCP formulations are prepared according to the method of Example 7. Table 3 shows the amounts of A-174, ethyl alcohol, acetic acid, and water to use to prepare various weights of solution, e.g. 25 g and 50 g. The amounts may be scaled proportionally to prepare different weights of solution as well.

Table 3 also shows the amount of solution to be sprayed onto 100.00 g of calcium phosphate. For instance, to prepare 3% weight coating, 29.00 g of solution is sprayed onto 100.00 g of calcium phosphate. The amounts may be scaled proportionally to prepare different coating weights onto different amounts of calcium phosphate as well at, for example, 10, 15, 20 and 25 wt % coating.

### Example 9

### Silanation with 4-aminobutyltriethoxysilane

100 g of 1-2 mm calcium phosphate was added to a mixing bowl. A 4-aminobutyltriethoxysilane solution was prepared with 12.5 g 4-aminobutyltriethoxysilane, 10 g ethyl alcohol, 1.25 g acetic acid, and 1.25 g water and then poured into a spray bottle. The spray bottle was weighed and the weight was recorded.

A 1% silicate beta-TCP solution was prepared as follows. The 4-aminobutyltriethoxysilane solution was sprayed onto 100.00 mg calcium phosphate while the glass was continually mixed. After 2-3 sprays, the spray bottle was weighed and the change in weight was recorded such that the weight of solution per spray was roughly determined. Additional 4-aminobutyltriethoxysilane solution was sprayed until the weight of the spray bottle was reduced by 7.83 g. After the 4-aminobutyltriethoxysilane solution has been applied, the glass was mixed for an additional 5-10 minutes, with continuous scraping of the walls and the bottom of the bowl.

A lid was placed on the mixing bowl and the treated calcium phosphate was incubated in an oven for 120 hours at 50° C. Following incubation, the treated glass was poured onto a drying tray and placed back into the oven at 50° C. The glass was dried for 1 week at 50° C. to evaporate residual ethanol and acetic acid. The silicated TCP was removed from the oven. ICP-MS and FTIR scans for the material were obtained to determine the amount of silica present.

### Example 10

### Silanation with 4-Aminobutyltriethoxysilane to Prepare Various Silicated TCP Formulations

TABLE 4

| | | | | |
|---|---|---|---|---|
| Material | % | MW | 25 g | 50 g |

4-aminobutyltriethoxysilane Solution

| | | | | |
|---|---|---|---|---|
| Silane | 50.00 | 235.4 | 12.5 | 25.00 |
| Ethyl Alcohol | 40.00 | 46.00 | 10 | 20.00 |
| Acetic Acid | 5.00 | 74.00 | 1.25 | 2.50 |
| Water | 5.00 | 18.00 | 1.25 | 2.50 |

Silicated TCP Formulations

| | | | | |
|---|---|---|---|---|
| wt % Coating | 0.1% | 1% | 3% | 5% |
| Calcium | 100.00 | 100.00 | 100.00 | 100.00 |
| Phosphate (g) | | | | |
| Solution (g) | 0.78 | 7.83 | 23.50 | 39.17 |

Various different silicated TCP formulations are prepared according to the method of Example 9. Table 4 shows the amounts of 4-aminobutyltriethoxysilane, ethyl alcohol, acetic acid, and water to use to prepare various weights of solution, e.g. 25 g and 50 g. The amounts may be scaled proportionally to prepare different weights of solution as well.

Table 4 also shows the amount of solution to be sprayed onto 100.00 g of calcium phosphate. For instance, to prepare 3% weight coating, 23.50 g of solution is sprayed onto 100.00 g of calcium phosphate. The amounts may be scaled proportionally to prepare different coating weights onto different amounts of calcium phosphate as well at, for example, 10, 15, 20 and 25 wt % coating.

### Example 11

### Silanation with Partially Hydrolyzed TEOS-Spray Application Method

Compositions were prepared using the silanation with partially hydrolyzed TEOS-spray apply method as follows:

TABLE 5

| | | | | |
|---|---|---|---|---|
| Material | % | MW | 25 g | 50 g |

TEOS Solution Formulation

| | | | | |
|---|---|---|---|---|
| TEOS | 50.00 | 208.33 | 12.5 | 25.00 |
| Ethyl Alcohol | 40.00 | 46.00 | 10 | 20.00 |
| Acetic Acid | 5.00 | 74.00 | 1.25 | 2.50 |
| Water | 5.00 | 18.00 | 1.25 | 2.50 |

Silicated TCP Formulations

| | | | | |
|---|---|---|---|---|
| wt % Coating | 0.1% | 1% | 3% | 5% |
| Calcium | 100.00 | 100.00 | 100.00 | 100.00 |
| Phosphate (g) | | | | |
| Solution (g) | 0.70 | 7.00 | 21.00 | 35.00 |

a. Weigh 100 g of 1-2 mm calcium phosphate into a mixing bowl.
b. Prepare the TEOS solution from the materials listed in the top half of the chart and pour the solution into a spray bottle. Weigh the spray bottle containing the solution and record the weight.
c. Spray apply the TEOS solution to the calcium phosphate while continually mixing the TCP. After 2-3 sprays, weigh the spray bottle and record the change in weight.
d. Continue to apply the TEOS solution until the change in weight is equivalent to the weight of TEOS solution listed in the table above (i.e., 7.00 g of solution for 1% silicate 13-TCP).
e. After the TEOS solution has been applied, continue mixing TCP for 5-10 minutes, occasionally scraping the walls and bottom of bowl.
f. Place a lid on the mixing bowl to and incubate the treated calcium phosphate in an oven for 120 hours at 50° C.
g. Following incubation, pour the treated TCP onto a drying tray and place the TCP back into oven at 50° C.
h. Dry the TCP for 1 week at 50° C. to burn off residual ethanol and acetic acid.
i. Remove the silicated TCP from the oven and obtain ICP-MS and FTIR scans for the material to determine the amount of silica present.

Various different silicated TCP formulations are prepared according to the method of Example 11. Table 5 shows the amounts of TEOS, ethyl alcohol, acetic acid, and water to use to prepare various weights of solution, e.g. 25 g and 50 g. The amounts may be scaled proportionally to prepare different weights of solution as well.

Table 5 also shows the amount of solution to be sprayed onto 100.00 g of calcium phosphate. For instance, to prepare 3% weight coating, 29.00 g of solution is sprayed onto 100.00 g of calcium phosphate. The amounts may be scaled proportionally to prepare different coating weights onto different amounts of calcium phosphate as well at, for example, 10, 15, 20 and 25 wt % coating.

### Example 12

### Silanation with Partially Hydrolyzed TEOS

Silanation with Partially Hydrolyzed TEOS
a. Prepare the partially hydrolyzed TEOS gel by combining 10 g TEOS, 1.5 g 0.1 M HCl, and 10 g EtOH in a nalgene jar.
b. Gently mix the solution and screw the lid on to the jar.
c. Incubate the jar in an oven set to 85° C. for 48 hours.
d. Weigh 100 g of 1-2 mm calcium phosphate into a mixing bowl.
e. For a 1% coating, dissolve 6 g of the partially hydrolyzed TEOS in 60 g of EtOH and 6 g of 0.1 M HCl. Pour the TEOS solution into a spray bottle. Weigh the spray bottle containing the solution and record the weight.
f. Spray apply the TEOS solution to the calcium phosphate while continually mixing the TCP. After 2-3 sprays, weigh the spray bottle and record the change in weight.
g. Continue to apply the TEOS solution until the change in weight is equivalent to the weight of TEOS solution listed in the table above (i.e., 7.00 g of solution for 1% silicate 13-TCP).
h. After the TEOS solution has been applied, continue mixing TCP for 5-10 minutes, occasionally scraping the walls and bottom of bowl.
i. Place a lid on the mixing bowl to and incubate the treated calcium phosphate in an oven for 120 hours at 50° C.
j. Following incubation, pour the treated TCP onto a drying tray and place the TCP back into oven at 50° C.
k. Dry the TCP for 1 week at 50° C. to burn off residual ethanol and acetic acid.
l. Remove the silicated TCP from the oven and obtain ICP-MS and FTIR scans for the material to determine the amount of silica present.

### Example 13

### Silanation with Silbond 50

Compositions were prepared by silanation with Silbond 50 as follows:

TABLE 6

| | | | | |
|---|---|---|---|---|
| Material | % | 25 g | 50 g | 100 g |

Silbond 50 Solution

| | | | | |
|---|---|---|---|---|
| Silbond 50 | 50.00 | 12.5 | 25.00 | 50.00 |
| Ethyl Alcohol | 25.00 | 6.25 | 12.50 | 25.00 |
| 0.1M HCl | 25.00 | 6.25 | 12.50 | 25.00 |

Silicated TCP Formulations

| | | | | |
|---|---|---|---|---|
| wt % Coating | 0.1% | 1% | 3% | 5% |
| Calcium | 100.00 | 100.00 | 100.00 | 100.00 |
| Phosphate (g) | | | | |
| Solution (g) | 0.43 | 4.35 | 13.04 | 21.74 |

a. Weigh 100 g of 1-2 mm calcium phosphate into a mixing bowl.
b. Prepare the Silbond 50 solution from the materials listed in the top half of the chart and pour the solution into a spray bottle. Weigh the spray bottle containing the solution and record the weight.
c. Spray apply the Silbond 50 solution to the calcium phosphate while continually mixing the TCP. After 2-3 sprays, weigh the spray bottle and record the change in weight.
d. Continue to apply the Silbond 50 solution until the change in weight is equivalent to the weight of TEOS solution listed in the table above (i.e., 7.00 g of solution for 1% silicate 13-TCP).
e. After the Silbond 50 solution has been applied, continue mixing TCP for 5-10 minutes, occasionally scraping the walls and bottom of bowl.
f. Place a lid on the mixing bowl to and incubate the treated calcium phosphate in an oven for 120 hours at 50° C.
g. Following incubation, pour the treated TCP onto a drying tray and place the TCP back into oven at 50° C.
h. Dry the TCP for 1 week at 50° C. to burn off residual ethanol.
i. Remove the silicated TCP from the oven and obtain ICP-MS and FTIR scans for the material to determine the amount of silica present.

Various different silicated TCP formulations are prepared according to the method of Example 13. Table 6 shows the amounts of Silbond 50, ethyl alcohol and hydrochloric acid to use to prepare various weights of solution, e.g. 25 g and 50 g. The amounts may be scaled proportionally to prepare different weights of solution as well.

Table 6 also shows the amount of solution to be sprayed onto 100.00 g of calcium phosphate. For instance, to prepare 3% weight coating, 29.00 g of solution is sprayed onto 100.00 g of calcium phosphate. The amounts may be scaled proportionally to prepare different coating weights onto different amounts of calcium phosphate as well at, for example, 10, 15, 20 and 25 wt % coating.

### Example 14

### Silanation with GPMES

Compositions were prepared by silanation with GPMES as follows:

TABLE 7

| | | | | |
|---|---|---|---|---|
| Material | % | MW | 25 g | 50 g |

(3-Glycidoxypropyl)dimethylethoxysilane (GPMES) Solution

| | | | | |
|---|---|---|---|---|
| GPMES | 50.00 | 218.37 | 12.5 | 25.00 |
| Ethyl Alcohol | 40.00 | 46.00 | 10 | 20.00 |
| Acetic Acid | 5.00 | 74.00 | 1.25 | 2.50 |
| Water | 5.00 | 18.00 | 1.25 | 2.50 |

Silicated TCP Formulations

| | | | | |
|---|---|---|---|---|
| wt % Coating | 0.1% | 1% | 3% | 5% |
| Calcium | 100.00 | 100.00 | 100.00 | 100.00 |
| Phosphate (g) | | | | |
| Solution (g) | 0.73 | 7.27 | 21.80 | 36.34 |

a. Weigh 100 g of 1-2 mm calcium phosphate into a mixing bowl.
b. Prepare the GPMES solution from the materials listed in the top half of the chart and pour the solution into a spray bottle. Weigh the spray bottle containing the solution and record the weight.
c. Spray apply the GPMES solution to the calcium phosphate while continually mixing the TCP. After 2-3 sprays, weigh the spray bottle and record the change in weight.
d. Continue to apply the GPMES solution until the change in weight is equivalent to the weight of GPMES solution listed in the table above (i.e., 7.27 g of solution for 1% silicated 13-TCP).
e. After the GPMES solution has been applied, continue mixing TCP for 5-10 minutes, occasionally scraping the walls and bottom of bowl.
f. Place a lid on the mixing bowl to and incubate the treated calcium phosphate in an oven for 120 hours at 50° C.
g. Following incubation, pour the treated TCP onto a drying tray and place the TCP back into oven at 50° C.
h. Dry the TCP for 1 week at 50° C. to burn off residual ethanol and acetic acid.
i. Remove the silicated TCP from the oven and obtain ICP-MS and FTIR scans for the material to confirm the amount of silica present.

Various different silicated TCP formulations are prepared according to the method of Example 14. Table 7 shows the amounts of GPMES, ethyl alcohol, acetic acid and water to use to prepare various weights of solution, e.g. 25 g and 50 g. The amounts may be scaled proportionally to prepare different weights of solution as well.

Table 7 also shows the amount of solution to be sprayed onto 100.00 g of calcium phosphate. For instance, to prepare 3% weight coating, 29.00 g of solution is sprayed onto 100.00 g of calcium phosphate. The amounts may be scaled proportionally to prepare different coating weights onto different amounts of calcium phosphate as well at, for example, 10, 15, 20 and 25 wt % coating.

### Example 15

### Silanation with A-174

Compositions prepared by silanation with A-174 were prepared as follows:

TABLE 8

| | | | | |
|---|---|---|---|---|
| Material | % | MW | 25 g | 50 g |

Methacryloxypropyltriethoxysilane (A-174) Solution

| | | | | |
|---|---|---|---|---|
| A-174 | 50.00 | 290.43 | 12.5 | 25.00 |
| Ethyl Alcohol | 40.00 | 46.00 | 10 | 20.00 |
| Acetic Acid | 5.00 | 74.00 | 1.25 | 2.50 |
| Water | 5.00 | 18.00 | 1.25 | 2.50 |

Silicated TCP Formulations

| | | | | |
|---|---|---|---|---|
| wt % Coating | 0.1% | 1% | 3% | 5% |
| Calcium | 100.00 | 100.00 | 100.00 | 100.00 |
| Phosphate (g) | | | | |
| Solution (g) | 0.97 | 9.67 | 29.00 | 48.33 |

a. Weigh 100 g of 1-2 mm calcium phosphate into a mixing bowl.
b. Prepare the A-174 solution from the materials listed in the top half of the chart and pour the solution into a spray bottle. Weigh the spray bottle containing the solution and record the weight.
c. Spray apply the A-174 solution to the calcium phosphate while continually mixing the TCP. After 2-3 sprays, weigh the spray bottle and record the change in weight.
d. Continue to apply the A-174 solution until the change in weight is equivalent to the weight of TEOS solution listed in the table above (i.e., 9.67 g of A-174 solution for 1% silicated β-TCP).
e. After the A-174 solution has been applied, continue mixing TCP for 5-10 minutes, occasionally scraping the walls and bottom of bowl.
f. Place a lid on the mixing bowl to and incubate the treated calcium phosphate in an oven for 120 hours at 50° C.
g. Following incubation, pour the treated TCP onto a drying tray and place the TCP back into oven at 50° C.
h. Dry the TCP for 1 week at 50° C. to burn off residual ethanol and acetic acid.
i. Remove the silicated TCP from the oven and obtain ICP-MS and FTIR scans for the material to confirm the amount of silica present.

Various different silicated TCP formulations are prepared according to the method of Example 15. Table 8 shows the amounts of A-174, ethyl alcohol, acetic acid and water to use to prepare various weights of solution, e.g. 25 g and 50 g. The amounts may be scaled proportionally to prepare different weights of solution as well.

Table 8 also shows the amount of solution to be sprayed onto 100.00 g of calcium phosphate. For instance, to prepare 3% weight coating, 29.00 g of solution is sprayed onto 100.00 g of calcium phosphate. The amounts may be scaled proportionally to prepare different coating weights onto different amounts of calcium phosphate as well at, for example, 10, 15, 20 and 25 wt % coating.

### Example 16

### Silanation with 4-aminobutyltriethoxysilane

Compositions were prepared with silanation with 4-aminobutyltriethoxysilane as follows:

TABLE 9

| | | | | |
|---|---|---|---|---|
| Material | % | MW | 25 g | 50 g |

4-aminobutyltriethoxysilane Solution

| | | | | |
|---|---|---|---|---|
| Silane | 50.00 | 235.4 | 12.5 | 25.00 |
| Ethyl Alcohol | 40.00 | 46.00 | 10 | 20.00 |
| Acetic Acid | 5.00 | 74.00 | 1.25 | 2.50 |
| Water | 5.00 | 18.00 | 1.25 | 2.50 |

Silicated TCP Formulations

| | | | | |
|---|---|---|---|---|
| wt % Coating | 0.1% | 1% | 3% | 5% |
| Calcium | 100.00 | 100.00 | 100.00 | 100.00 |
| Phosphate (g) | | | | |
| Solution (g) | 0.78 | 7.83 | 23.50 | 39.17 |

a. Weigh 100 g of 1-2 mm calcium phosphate into a mixing bowl.
b. Prepare the silane solution from the materials listed in the top half of the chart and pour the solution into a spray bottle. Weigh the spray bottle containing the solution and record the weight.
c. Spray apply the silane solution to the calcium phosphate while continually mixing the TCP. After 2-3 sprays, weigh the spray bottle and record the change in weight.
d. Continue to apply the silane solution until the change in weight is equivalent to the weight of silane solution listed in the table above (i.e., 7.83 g of solution for 1% silicated 13-TCP).
e. After the TEOS solution has been applied, continue mixing TCP for 5-10 minutes, occasionally scraping the walls and bottom of bowl.
f. Place a lid on the mixing bowl to and incubate the treated calcium phosphate in an oven for 120 hours at 50° C.
g. Following incubation, pour the treated TCP onto a drying tray and place the TCP back into oven at 50° C.
h. Dry the TCP for 1 week at 50° C. to burn off residual ethanol and acetic acid
i. Remove the silicated TCP from the oven and obtain ICP-MS and FTIR scans for the material to confirm the amount of silica present.

Various different silicated TCP formulations are prepared according to the method of Example 16. Table 9 shows the amounts of 4-aminobutyltriethoxysilane, ethyl alcohol, acetic acid and water to use to prepare various weights of solution, e.g. 25 g and 50 g. The amounts may be scaled proportionally to prepare different weights of solution as well.

Table 9 also shows the amount of solution to be sprayed onto 100.00 g of calcium phosphate. For instance, to prepare 3% weight coating, 29.00 g of solution is sprayed onto 100.00 g of calcium phosphate. The amounts may be scaled proportionally to prepare different coating weights onto different amounts of calcium phosphate as well at, for example, 10, 15, 20 and 25 wt % coating.

Samples prepared in accordance with Examples 11 and 13 were tested under ASTM D4698 for weight percent of calcium, phosphorous, and silicon. Samples labeled “SILBOND B” and “SILBOND C” were prepared in accordance Example 13 with a target Silicon content of 3% and 1% respectively. Samples labeled “TEOS 4” and TEOS 5″ were prepared in accordance with Example 11 with a target Silicon content of 1%. The following results were obtained:

TABLE 10

| | | Sample Concentration | | Minimum Reporting Limit | |
|---|---|---|---|---|---|
| | | | Parts per | | Parts per |
| Client | | Weight | Million | Weight | Million |
| Sample ID | Analyte | Percent (%) | (PPM) mg/kg | Percent (%) | (PPM) mg/kg |
| SILBOND B | Calcium | 33.3 | 333000 | 1.24 | 12400 |
| SILBOND B | Phosphorus | 17.4 | 174000 | 1.24 | 12400 |
| SILBOND B | Silicon | 2.73 | 27300 | 0.994 | 9940 |
| SILBOND C | Calcium | 33.4 | 334000 | 1.22 | 12200 |
| SILBOND C | Phosphorus | 17.3 | 173000 | 1.22 | 12200 |
| SILBOND C | Silicon | 1.19 | 11900 | 0.978 | 9780 |
| TEOS 4 | Calcium | 33.6 | 336000 | 1.24 | 12400 |
| TEOS 4 | Phosphorus | 17.6 | 176000 | 1.24 | 12400 |
| TEOS 4 | Silicon | 1.01 | 10100 | 0.995 | 9950 |
| TEOS 5 | Calcium | 35.2 | 352000 | 1.24 | 12400 |
| TEOS 5 | Phosphorus | 18.3 | 183000 | 1.24 | 12400 |
| TEOS 5 | Silicon | 1.20 | 12000 | 0.991 | 9910 |

### Example 17

### Preparation of Sol-Gel Glass

Sol Gel Bioactive glasses were prepared with the compositions set forth in Table 11 and as described in 1-1 through 1-6 below:

TABLE 11
Compositions of Sol-gel Bioactive Glasses

| | SiO₂ | CaO | P₂O₅ | Na₂O |
|---|---|---|---|---|
| Sample ID | (wt. %) | (wt. %) | (wt. %) | (wt. %) |
| 45S5 (melt) | 45 | 24.5 | 6 | 24.5 |
| 45S5 (Sol-gel) | 45 | 24.5 | 6 | 24.5 |
| 58S | 58 | 33 | 9 | 0 |
| 77S | 77 | 14 | 9 | 0 |
| 100S | 100 | 0 | 0 | 0 |

Preparation of 1-1. 100S gel (Comparative—no Na, Ca, or P source): the gel was prepared by mixing D. I. water, HCl, TEOS (Tetraethoxysilane) followed by mixing for 60 minutes to facilitate the completion of hydrolysis reaction. Then, the mixture was applied to the calcium salt composition and dried.

Preparation of 1-2. 77S gel (Comparative—no Na source): the gel was prepared by mixing D. I. water, HCl, TEOS (Tetraethoxysilane) for 30 minutes, adding TEP (Triethylphosphate) into the solution and mixing for another 20 minutes, then adding CaNO₃.4H₂O (Calcium Nitrate tetra-hydrate) while mixing for an additional 60 minutes to complete the dissolution of the Calcium Nitrate. Then, the mixture was applied to the calcium salt and dried as need to form the glass coating.

Preparation of 1-3 (Comparative—no Na source). 58S gel: the gel was prepared by mixing D. I. water, HCl, TEOS (Tetraethoxysilane) for 30 minutes, adding TEP (Triethylphosphate) into the solution and mixing another 20 minutes, then adding CaNO3.4H2O (Calcium Nitrate tetra-hydrate) while mixing for an additional 60 minutes to complete the dissolution of the Calcium Nitrate. Then, the mixture was applied to the calcium salt and dried as need to form the glass coating.

Preparation of 1-4. 45S5 gel#1 (Includes sodium ethoxide as Na source): the gel was prepared by mixing half the amount of D. I. water, HCl, TEOS (Tetraethoxysilane) for 30 minutes, adding TEP (Triethylphosphate) into the solution and mixing another 20 minutes, then adding the rest of D. I. water, Calcium Methoxide, and Sodium Ethoxide, while mixing for 60 minutes to complete the hydrolysis reaction. Then, the mixture was applied to the calcium salt and dried as need to form the glass coating.

Preparation of 1-5. 45S5 gel#2 (Includes NaCl as Na source): the gel was prepared by mixing D. I. water, HCl, TEOS (Tetraethoxysilane) for 30 minutes, adding TEP(triethylphosphate) into the solution and mixing another 20 minutes, then adding CaNO₃.4H₂O (Calcium Nitrate tetra-hydrate) and NaCl while mixing for an additional 60 minutes to complete the dissolution of the Calcium Nitrate and NaCl. Then, the mixture was applied to the calcium salt and dried as need to form the glass coating.

Preparation of 1-6. 45S5 gel#3 (Comparative—includes sodium nitrate as Na source): the gel was prepared by mixing D. I. water, HCl, TEOS (Tetraethoxysilane) for 30 minutes, adding TEP(triethylphosphate) into the solution and mixing another 20 minutes, then adding CaNO₃.4H₂O (Calcium Nitrate tetra-hydrate) and NaNO₃ (Sodium Nitrate), while mixing for an additional 60 minutes to complete the dissolution of the Calcium Nitrate and Sodium Nitrate. Then, the mixture was transferred into a polypropylene mold for aging at 60° C. for 55 hours. After aging, the precipitation could be seen visually. After aging, the gel was applied to the calcium salt and dried as need to form the glass coating.

The porous structure data was obtained from the foregoing compositions as noted in Table 12.

TABLE 12

| | Specific Surface | Pore Size |
|---|---|---|
| | Area of coating | Diameter of coating |
| Sample ID | m²/gram | (Angstroms) |
| 45S5(Melt) | 0.1 | 0 |
| 45S5(Sol-gel) | 31 | 98 |
| 58S | 166 | 96 |
| 77S | 414 | 30 |
| 100S | 561 | 40 |

1. A composition comprising calcium salt, silica and a metallic material having an atomic mass greater than 45 and less than 205,
wherein the silica is in the form of a silicate that is adsorbed only onto a surface of the calcium salt and is not incorporated into the structure of the calcium salt, and
wherein the composition is bioactive.

2. The composition of claim 1, wherein the silica is an organosilane, a sol-gel composition, a solution of silicated salt, a combination thereof or other silica-containing composition.

3. The composition of claim 1, wherein the calcium salt is selected from calcium carbonate, calcium borate, calcium sulfate, calcium phosphate, calcium silicate or beta calcium triphosphate.

4. The composition of claim 1, wherein the metallic material is selected from the group consisting of gold, silver, platinum, copper, palladium, iridium, strontium, cerium, an isotope, an alloy or a combination thereof.

5. The composition of claim 1, wherein a weight ratio of the metallic material is about 0.001%-20% relative to the weight of the composition.

6. The composition of claim 1, wherein a weight ratio of the metallic material is 0.001%-10% relative to the weight of the composition.

7. The composition of claim 1, wherein the metallic material is dispersed into the silica.

8. The composition of claim 1, wherein the metallic material forms a coating on the surface of the calcium salt.

9. The composition of claim 1, wherein the metallic material forms a coating over the silicate that is adsorbed onto a surface of the calcium salt.

10. The composition of claim 1, wherein the composition is osteoinductive.

11. The composition of claim 1, wherein a sufficient quantity of silica is present to reduce the resorption rate of calcium.

12. The composition of claim 1, wherein the adsorbed silica is effective to reduce the rate of adsorption of calcium.

13. The composition of claim 12, wherein the adsorbed silica forms a layer that is effective to reduce the rate of adsorption of calcium carbonate.

14. The composition of claim 12, wherein the adsorbed silica forms a layer that is effective to reduce the rate of adsorption of calcium borate.

15. The composition of claim 12, wherein the adsorbed silica forms a layer that is effective to reduce the rate of adsorption of calcium sulfate.

16. The composition of claim 12, wherein the adsorbed silica forms a layer that is effective to reduce the rate of adsorption of calcium phosphate.

17. The composition of claim 12, wherein the adsorbed silica forms a layer that is effective to reduce the rate of adsorption of beta calcium triphosphate.

18. The composition of claim 1, wherein the calcium and silica are effective to stimulate osteoblast differentiation and osteoblast proliferation.

19. The composition of claim 1, wherein a ratio of silica and the composition is from 0.01 wt % to 50 wt %.

20. The composition of claim 1, wherein a ratio of silica and the composition is from 1 wt % to 25 wt %.

21. The composition of claim 1, wherein the silicate is substituted with a functional group.

22. The composition of claim 1, wherein the composition conducts an electrical current.

23. The composition of claim 1, wherein the composition promotes more rapid wound healing as compared to a composition without the metallic material.

24. The composition of claim 2, wherein the organosilane is selected from a group consisting of γ-methacryloxypropyltrimethoxysilane, (3-glycidoxypropyl)-dimethyl-ethoxysilane, partially hydrolyzed tetraethyl orthosilicate, Silbond, 4-aminobutyltriethoxysilane, (3-aminopropyl)-triethoxysilane, (3-aminopropyl)-diethoxy-methylsilane, (3-aminopropyl)-dimethyl-ethoxysilane, (3-aminopropyl)-trimethoxysilane, (3-mercaptopropyl)-trimethoxysilane, and a combination thereof.

25. The composition of claim 1, wherein the silica further comprises at least one of monovalent, divalent, trivalent metal ion, or anionic specie thereof.

26. A method to stimulate osteoblast differentiation comprising contacting an osteoblast with the composition of claim 1.

27. A method to stimulate osteoblast proliferation comprising contacting an osteoblast with the composition of claim 1.

28. A method of regenerating bone comprising contacting the bone at or near a site of a bone defect with a composition of claim 1.

29. A method of achieving critical concentrations of calcium ions and silicate ions in a bone defect by contacting a bone at or near a site of the bone defect with a composition of claim 1.

30. A composition comprising calcium salt, silica, and a metallic material having an atomic mass greater than 45 and less than 205,
wherein the silica is in an organic or inorganic form selected from the group consisting of an organosilane, a sol-gel composition, a solution of silicated salt, a combination thereof and other silica-containing composition and is adsorbed only onto a surface of the calcium salt,
wherein the silica is not incorporated into the structure of the calcium salt, and wherein the composition is bioactive.

31. The composition of claim 30, wherein the organosilane is selected from a group consisting of γ-methacryloxypropyltrimethoxysilane, (3-glycidoxypropyl)-dimethyl-ethoxysilane, partially hydrolyzed tetraethyl orthosilicate, Silbond, 4-aminobutyltriethoxysilane, (3-aminopropyl)-triethoxysilane, (3-aminopropyl)-diethoxy-methylsilane, (3-aminopropyl)-dimethyl-ethoxysilane, (3-aminopropyl)-trimethoxysilane, (3-mercaptopropyl)-trimethoxysilane, and a combination thereof.

32. The composition of claim 30, wherein the metallic material is selected from the group consisting of gold, silver, platinum, copper, palladium, iridium, strontium, cerium, an isotope, an alloy or a combination thereof.

33. The composition of claim 30, wherein a weight ratio of the metallic material is about 0.001%-20% relative to the weight of the composition.

34. The composition of claim 30, wherein a weight ratio of the metallic material is 0.001%-10% relative to the weight of the composition.

35. The composition of claim 30, wherein the composition conducts an electrical current.

36. The composition of claim 30, wherein the metallic material is dispersed into the silica.

37. The composition of claim 30, wherein the metallic material forms a coating on the surface of the calcium salt.

38. The composition of claim 30, wherein the metallic material forms a coating over the silica that is adsorbed onto the surface of the calcium salt.
